(19) United States
(12) Patent Application Publication
(10) Pub. No.: US 2026/0364304 A1
(43) Pub. Date: Nov. 19, 2026

(54) Suicide treatment room: System and Method for Treating Suicidal Ideations Through Controlled Environmental Isolation and Simulated Mortality Exposure.

(71) Applicant: Mariam Bekhit, East Brunswick, NJ (US)

(72) Inventor: Mariam Bekhit, East Brunswick, NJ (US)

(21) Appl. No.: 19/207,197

(22) Filed: May 13, 2025

(51) Int. Cl. A61M 21/00

(52) U.S. Cl. CPC A61M 21/0094; A61M 2021/0027; A61M 2021/005

(57) ABSTRACT

This invention pertains to a novel method for preventing suicidality in at-risk individuals through a structured protocol involving controlled isolation and simulated funeral imagery. The method is designed to facilitate a therapeutic environment that allows patients to confront and process their emotions safely. By integrating elements of controlled isolation and guided visualization, this approach aims to reduce suicidal ideation and promote mental well-being. The protocol has been developed with a focus on safety, ethical considerations, and clinical efficacy, offering a potentially transformative tool for mental health practitioners.

### FIELD OF THE INVENTION

This invention relates to the treatment of mental health disorders, and more particularly, to systems and methods for reducing suicidal ideation through structured, immersive psychological interventions for both adults and minors.

### BACKGROUND OF THE INVENTION

The method of treating suicidality presented in this application involves two phases:
1. A funeral simulation that allows the patient to experience the emotional consequences of their death on loved ones.
2. A post-death consciousness simulation, inducing a psychological experience of irreversible loss and disembodied isolation, aiming to evoke emotional insight and discourage self-destructive behavior.

This system is designed to be safe for patients as young as 10 years old, with appropriate modifications for developmental sensitivity.

### SUMMARY OF THE INVENTION

The invention provides a novel system and method for reducing suicidal ideation, consisting of:
A controlled environment where the patient is secluded in a minimalistic, dimly lit room or virtual space.
Simulated funeral and death exposure via audiovisual content.
Post-death consciousness simulation wherein the patient experiences disembodied isolation, attempts to reconnect with loved ones, and the realization of irreversible loss and the persistence of consciousness.
Pediatric-specific adaptations: pre-screening, modified content, and parental consent and involvement.

This approach can be delivered in clinical settings, hospital settings, or virtual reality, and is applicable to individuals aged 10 years and older.

1. A method of reducing suicidal ideation, comprising:
placing a patient in a non-personalized, enclosed treatment space;
exposing the patient to audiovisual content simulating their own funeral;
exposing the patient to a post-death consciousness sequence in which they experience irreversible disconnection and the persistence of self-awareness;
allowing time for emotional reflection; and
monitoring the patient for therapeutic response.

2. The method of claim 1, wherein the patient is a minor aged 10 years or older, and the audiovisual content is adapted for developmental appropriateness with consent from a legal guardian.

3. The method of claim 1, wherein the audiovisual content includes a simulated post-mortem burial and decay;
transition to a disembodied state in an unresponsive environment;
failed efforts to reconnect with the living world;
realization of permanent isolation and loss of agency.

4. The method of claim 3, wherein the patient attempts to destroy their energy form and discovers that self-annihilation is impossible.

5. The method of claim 3, wherein the virtual sequence is delivered through a VR headset or immersive display.

6. A system for implementing the method of claim 1, comprising:
a secluded treatment environment;
a media delivery unit;
a protocol guide for mental health clinicians;
pediatric-specific modifications for developmental appropriateness.
